(19) United States
(12) Patent Application Publication
(10) Pub. No.: US 2004/0191214 A1
(43) Pub. Date: Sep. 30, 2004

(54) Nucleoside vaccine adjuvants

(76) Inventors: Johnson Lau, Newport Beach, CA (US); Zhi Hong, Aliso Viejo, CA (US)

Correspondence Address: BROWN, RAYSMAN, MILLSTEIN, FELDER & STEINER LLP, 900 THIRD AVENUE, NEW YORK, NY, 10022, US

(21) Appl. No.: 10/475,718

(22) PCT Filed: Jun. 14, 2002
(86) PCT No.: PCT/US02/18724

(30) Foreign Application Priority Data
Jun. 15, 2001 (US) 60298551

(51) Int. Cl. A61K 39/00; A61K 38/19

(52) U.S. Cl. 424/85.1; 424/185.1; 514/44

(57) ABSTRACT

Contemplated methods and compositions include a pharmacological composition comprising an antigen and a nucleoside adjuvant that modulates the balance between Type 1 response and Type 2 response in a lymphocyte, wherein the nucleoside is not an 8-substituted guanine nucleoside. In preferred aspects, contemplated adjuvants stimulate Type 1 response and thereby increase a T-cell response, while contemplated adjuvants may also stimulate a Type 2 response and thereby increase a B-cell response. Preferred adjuvants may further comprise a CpG-dinucleotide and/or a CpG-containing oligonucleotide in a synergistic amount.

### FIELD OF THE INVENTION

The field of the invention is vaccine adjuvants.

### BACKGROUND OF THE INVENTION

Immunizations are generally a relatively simple procedure and typically include presentation of an antigen (e.g., attenuated virus, bacterial membrane preparation, synthetic peptides, etc.) to an immune system. While administration of some antigens is often sufficient to achieve a desired immunity, other antigens may require co-administration of the antigen with an adjuvant. There are numerous adjuvants known in the art, and one frequently used adjuvant is Freund's complete adjuvant. Where toxicity or viscosity of Freunds complete adjuvant is problematic, it is also known to employ an alternative adjuvant, comprising oil-in-water emulsion containing detoxified endotoxin and mycrobacterial cell wall components in 2% squalene, or a Listeria cell wall preparation (see e.g., U.S. Pat. No. 6,086,898 to DeKruyff et al.).

Although Freunds adjuvant and alternative toxin-containing adjuvants often result in relatively high titers of antigen specific antibodies, such adjuvants tend to produce a significant inflammatory reaction, which may be especially undesirable in humans. To reduce the incidence or severity of inflammatory reactions, aluminum hydroxide may be employed as an adjuvant. Aluminum hydroxide has the advantage of being especially suitable, which can be significant when relatively strong antigens are presented to the immune system. One disadvantage, however, is that aluminum hydroxide tends to produce significantly lower titers, especially when combined with moderate to weak antigens. Moreover, certain aluminum compounds have been thought to be involved in neurological damage associated with Alzheimer's Disease (AD).

Alternatively, antigens may be presented to the immune system as co-precipitates of the amino acid L-tyrosine. While co-precipitation of an antigen with L-tyrosine to produce the immunization cocktail is relatively simple, the preparation typically requires manipulation that often increases the cost of manufacturing, and may result in decreased antigenic activity.

In other approaches, Ag-modified saponin/cholesterol micelles forming cage-like structures may be employed as carriers for antigens. Such compositions are typically transported to the draining lymph nodes, and quantities of antigen as low as 1 μg have been shown to elicit a significant immune response. However, such compositions may be difficult to prepare, and tend to be relatively expensive on a large scale.

In further approaches, as described in U.S. Pat. No. 5,876,966 to Reed, synthetic peptides can be co-administered with an antigen, thereby stimulating a Th1 response and IL-12 production in a patient. Although Reed's approach is relatively effective in eliciting an immune response, synthetic peptides are often expensive. Moreover, synthetic peptides may be poorly tolerated by at least some patients. Alternatively, IL-12 can be co-administered as an adjuvant as described in U.S. Pat. No. 5,976,539 to Scott, et al. However, production and purification of IL-12 is relatively expensive, and may induce undesirable side effects in at least some patients.

In still further approaches, unmethylated CpG dinucleotides within the context of certain flanking sequences (CpG motifs) have been shown to stimulate both innate and specific immune responses, which has been demonstrated for synthetic as well as naturally occurring sequences. For example, it was previously shown that CpG DNA induces stimulation of B cells to proliferate and secrete antibodies, IL-6 and IL-12, and protects such cells under certain circumstances from apoptosis (see e.g., Krieg et al., in Nature (1995), Apr 6; 374(6522):546-91995). Furthermore, CpG DNA is thought to enhance expression of class II MHC and B7 co-stimulatory molecules (Sparwasser et al., in Eur J Immunol. (1998) Jun; 28(6):2045-54), thereby leading to improved antigen presentation.

These findings were further confirmed by the observation that immunization of animals against various antigens using CpG oligos as adjuvants induces relatively strong Th1-like responses as indicated by highly cytotoxic T lymphocytes (CTL), high levels of IgG2a antibodies, and predominantly Th1 cytokines (e.g., IL-12 and IFN-gamma, but not IL-4 or 5; see e.g., Klinman et al., in Proc Natl Acad Sci U S A. (1996) Apr 2; 93(7):2879-83 1996, Roman et al., in Allergy (1998); 53(45 Suppl):93-7, Chu et al., in J Exp Med. (1997) Nov 17; 186(10):1623-31, Lipford et al., in Eur J Immunol (1997) Sep; 27(9):2340-4). Interestingly, non-CpG oligonucleotides have also been employed as Th2-stimulating adjuvants as described in published U.S. Patent application 20010044416 to McCluskie et al. While such CpG and non-CpG oligonucleotides may present relatively potent adjuvants, various disadvantages remain. Among other things, oligonucleotides tend to be relatively instable, especially in blood or serum. Moreover, and depending on the particular composition, undesirable adverse effects may occur.

Alternatively, selected 8-substituted guanine derivatives have been proposed to modulate cellular responses of an animal towards various antigens as described in U.S. Pat. Nos. 5,317,013, 5,166,141, 5,147,636, 4,948,730, 4,849,411, 4,643,992, or 4,539,205, all to Goodman et al. While some of these selected 8-substituted guanine derivatives have demonstrated significant adjuvant effect in combination with various antigens, the use of these selected 8-substituted guanine derivatives may pose numerous problems. Most significantly, many 8-substituted guanine derivatives may act as DNA/RNA polymerase inhibitors, thereby rendering these compounds potentially toxic and/or mutagenic.

Although there are various adjuvants and immunizing compositions known in the art, most or all of them suffer from at least some significant disadvantage. Therefore, there is still a need to provide improved methods and compositions for adjuvants and immunizing compositions.

### SUMMARY OF THE INVENTION

The present invention is directed to methods and compositions for adjuvants and immunizing compositions comprising an antigen and an adjuvant, wherein the adjuvant includes a nucleoside that modulates a balance between a Type 1 cytokine response and a Type 2 cytokine response in a lymphocyte., and wherein the nucleoside is not an 8-substituted guanine nucleoside.

In one aspect of the inventive subject matter, the antigen is a viral antigen, a bacterial antigen, or a tumor-specific antigen. Especially preferred adjuvants include nucleoside analogs such as Ribavirin, Levovirin, and Viramidine, and it is further contemplated that the nucleoside analogs or other suitable adjuvant may be combined with CpG oligonucleotides, CpG dinucleotides, or derivatives of such oligo- and/or dinucleotides to achieve an improved immune response.

In another aspect of the inventive subject matter, the modulation of the balance comprises a relative increase in the Type 1 response over the Type 2 response. It is further contemplated that nucleoside analogs that stimulate a Th1 response may promote T-cell activated responses to an immunogen, while nucleoside analogs that stimulate a Th2 response may promote B-cell activated responses to an immunogen.

Various objects, features, aspects and advantages of the present invention will become more apparent from the following detailed description of preferred embodiments of the invention.

### DETAILED DESCRIPTION

The term “nucleoside” as used herein refers to all compounds in which a heterocyclic base is covalently coupled to a sugar, and an especially preferred coupling of the nucleoside to the sugar includes a Cl′-(glycosidic) bond of a carbon atom in a sugar to a carbon- or heteroatom (typically nitrogen) in the heterocyclic base. The term “nucleoside analog” as also used herein refers to all nucleosides in which the sugar is not a ribofuranose and/or in which the heterocyclic base is not a naturally occurring base (e.g., A, G, C, T, I, etc.). Similarly, the term “nucleotide” refers to a nucleoside to which a phosphate group is covalently coupled to the sugar. Likewise, the term “nucleotide analog” refers to a nucleoside analog to which a phosphate group is coupled to the sugar.

As further used herein, the term “heterocyclic base” refers to any compound in which a plurality of atoms form a ring via a plurality of covalent bonds, and wherein the ring includes at least one atom other than a carbon atom. Particularly contemplated heterocyclic bases include 5- and 6-membered rings with nitrogen, sulfur, and/or oxygen as the non-carbon atom (e.g., imidazole, pyrrole, triazole, dihydropyrimidine). Further contemplated heterocylces may be fused (ie., covalently bound) to another ring or heterocycle, and are thus termed “fused heterocyclic base”. Especially contemplated fused heterocyclic bases include a 5-membered ring fused to a 6-membered ring (e.g., purine, pyrrolo[2,3-d]pyrimidine), and a 6-membered ring fused to another 6-membered or higher ring (e.g., pyrido[4,5-d]pyrimidine, benzodiazepine). Examples of these and further preferred heterocyclic bases are given below. Still further contemplated heterocyclic bases may be aromatic, or may include one or more double or triple bonds. Moreover, contemplated heterocyclic bases and fused heterocycles may further include substituents in one or more positions (see below).

As further used herein, the term “sugar” refers to all carbohydrates (i.e., molecules with the formula CnH₂nOn with n typically between 4 and 12) and derivatives thereof, wherein particularly contemplated derivatives include deletion, substitution or addition of a chemical group or atom in the sugar. For example, especially contemplated deletions include 2′-deoxy and/or 3′-deoxy sugars. Especially contemplated substitutions include replacement of the ring-oxygen with sulfur or methylene, or replacement of a hydroxyl group with a halogen, an amino-, sulfhydryl-, or methyl group, and especially contemplated additions include methylene phosphonate groups. Further contemplated sugars also include sugar analogs (i.e., not naturally occurring sugars), and particularly carbocyclic ring systems. The term “carbocyclic ring system” as used herein refers to any molecule in which a plurality of carbon atoms form a ring, and in especially contemplated carbocyclic ring systems the ring is formed from 3, 4, 5, or 6 carbon atoms.

A still further used herein, the term “substituted” as used herein refers to a replacement of an atom or chemical group (e.g., H, NH₂, or OH) with a functional group, and particularly contemplated functional groups include nucleophilic groups (e.g., —NH₂, —OH, —SH, —NC, etc.), electrophilic groups (e.g., C(O)OR, C(X)OH, etc.), polar groups (e.g., —OH), non-polar groups (e.g., aryl, alkyl, alkenyl, alkynyl, etc.), ionic groups (e.g., —NH₃⁺), and halogens (e.g., —F, —Cl), and all chemically reasonable combinations thereof Thus, the term “functional group” and substituent are used interchangeably herein and refer to a nucleophilic group (e.g., —NH₂, —OH, —SH, —NC, —CN etc.), an electrophilic group (e.g., C(O)OR, C(X)OH, C(Halogen)OR, etc.), a polar group (e.g., —OH), a non-polar group (e.g., aryl, alkyl, alkenyl, alkynyl, etc.), an ionic group (e.g., —NH₃⁺), and/or a halogen.

It should further be particularly appreciated that the terms nucleoside, nucleotide, nucleoside analog, and/or nucleotide analog also includes all prodrug forms of a nucleoside, nucleotide, nucleoside analog, and/or nucleotide analog, wherein the prodrug form may be activated/converted to the active drug/nucleoside, nucleotide, nucleoside analog, and/or nucleotide analog in one or more than one step, and wherein the activation/conversion of the prodrug into the active drug/nucleoside, nucleotide, nucleoside analog, and/or nucleotide analog may occur intracellularly or extracellularly (in a single step or multiple steps). Especially contemplated prodrug forms include those that confer a particular specificity towards a diseased or infected cell or organ, and exemplary contemplated prodrug forms are described in “Prodrugs” by Kenneth B. Sloan (Marcel Dekker; ISBN: 0824786297), “Design of Prodrugs” by Hans Bundgaard (ASIN: 044480675X), or in copending U.S. application No. 09/594410, filed Jun. 16, 2000, all of which are incorporated by reference herein. Particularly suitable prodrug forms of the above compounds may include a moiety that covalently coupled to at least one of the C2′-OH, C3′-OH, and C5′-OH, wherein the moiety is preferentially cleaved from the compound in a target cell (e.g., Hepatocyte) or a target organ (e.g., liver). While not limiting to the inventive subject matter, it is preferred that cleavage of the prodrug into the active form of the drug is mediated (at least in part) by a cellular enzyme, and particularly receptor, transporter and cytochrome-associated enzyme systems (e.g., CYP-system).

Still further particularly preferred prodrugs include those described by Renze et al. in Nucleosides Nucleotides Nucleic Acids 2001 Apr-Jul;20(4-7):9314, by Balzarini et al. in Mol Pharmacol 2000 Nov;58(5):928-35, or in U.S. Pat. No. 6,312,662 to Erion et al., U.S. Pat. No. 6,271,212 to Chu et al., U.S. Pat. No. 6,207,648 to Chen et al., U.S. Pat. No. 6,166,089 and U.S. Pat. No. 6,077,837 to Kozak, U.S. Pat. No. 5,728,684 to Chen, and published U.S. Application with the number 20020052345 to Erion, all of which are incorporated by reference herein. Alternative contemplated prodrugs include those comprising a phosphate and/or phosphonate non-cyclic ester, and an exemplary collection of suitable prodrugs is described in U.S. Pat. No. 6,339,154 to Shepard et al., U.S. Pat. No. 6,352,991 to Zemlicka et al., and U.S. Pat. No. 6,348,587 to Schinazi et al. Still further particularly contemplated prodrug forms are described in FASEB J. 2000 Sep;14(12):1784-92, Pharm. Res. 1999, Aug 16:8 1179-1185, and Antimicrob Agents Chemother 2000, Mar 44:3 477483, all of which are incorporated by reference herein.

As still further used herein, the term “8-substituted guanine nucleoside” refers to compounds that include a guanine heterocyclic base with a chemical modification at the 8-position, wherein the guanine portion is covalently coupled to a sugar. Compounds that fall under the scope of this definition are described in U.S. Pat. Nos. 5,317,013, 5,166,141, 5,147,636, 4,948,730, 4,849,411, 4,643,992, or 4,539,205, all to Goodman et al., and all incorporated by reference herein.

As yet further used herein, the term “antigen” refers to a substance that when presented to an immune system stimulates production of an antibody (e.g., toxins, bacteria, foreign blood cells, cells of transplanted organs, etc.) against the substance, or comprises a non-self (relative to the immune system host) component, which may or may not stimulate production of an antibody against the non-self component. Furthermore, as used herein, the term “adjuvant” refers to a compound or composition that agent that increases an antigenic response in an immune system against an antigen, wherein the increase in antigenic response my be measured, among other things, by an increase in titer or affinity in antigen-specific antibodies, or an increase in a cytotoxic T-cell response.

Finally, as used herein, the term “modulate a cytokine balance between a Type 1 response and a Type 2” means to bring about a quantitative change in expression and/or secretion of at least one of a Type 1 (e.g., IL-2, TNF-beta, or INF-gamma) and/or Type 2 cytokine (e.g., IL4, IL-5, IL-6, or IL-10). Thus, the term “modulate a cytokine balance between a Type 1 response and a Type 2” particularly includes an increase of expression and/or secretion of at least one of a Type 1 cytokine and/or a decrease of expression and/or secretion of at least one of a Type 2 cytokine (or vice versa). Modulation of a cytokine balance may readily be ascertained by a person of ordinary skill in the art without undue experimentation. For example, numerous protocols are well known in the art to determine (qualitatively and quantitatively) one or more cytokines (see e.g., Cytokine Cell Biology: A Practical Approach by Frances R. Balkwill; Oxford University Press; ISBN: 0199638594; 3rd edition (Jan. 15, 2001), or The Cytokine Handbook by Angus Thomson; Academic Press; ISBN: 0126896623; 3rd edition (Jul. 15, 1998)).

Contemplated Adjuvant Compounds

It is contemplated that all compounds that modulate the relative balance between a Type 1 response and a Type 2 response are suitable for use in conjunction with the teachings presented herein. Especially contemplated adjuvant compounds include nucleosides, nucleotides, and their respective analogs, in both D-, and L-configuration. For example, particularly contemplated nucleoside and nucleotide analogs include compounds in which either the base portion and/or the sugar portion of the nucleoside/nucleotide are modified. Furthermore, it should be recognized that prodrug forms of contemplated compounds are also contemplated.

Particularly contemplated adjuvant compounds include nucleosides in which the heterocyclic base comprises a diazole or triazole moiety that may or may not be further modified with various substituents and/or functional groups. Further especially preferred nucleosides will include a carbohydrate sugar moiety that is most preferably a ribofuranose in D- or L-configuration. For example, particularly suitable adjuvant compounds include Ribavirin (1-beta-D-ribofuranosyl-1,2,4-triazole-3-carboxamide, Formula 1), Levovirin (1-beta-L-ribofuranosyl-1,2,4-triazole-3-carboxamide,, Formula 2), and Viramidine (1-beta-D-ribofuranosyl-1,2,4-triazole-3-carboxamidine, Formula 3).

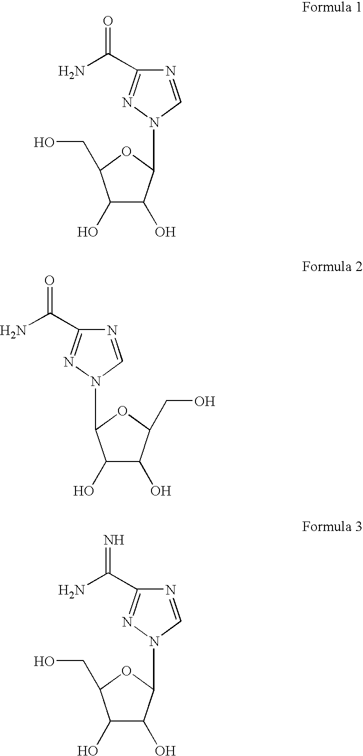

Variants and prodrug forms on these compounds are disclosed in copending U.S. application Ser. No. 09/594410, filed Jun. 16, 2000, copending PCT application number PCT/USOO/34605, filed Dec. 19, 2000, and copending PCT application number PCT/USO1/40148, filed Feb. 15, 2001, all of which are incorporated by reference herein.

Synthesis of triazole-containing nucleosides is well known in the art and is described, for example, in U.S. Pat. No. 4,138,547 to Christensen et al., incorporated by reference herein. In other especially preferred aspects, the base moiety comprises a Pyrido[2,3-d]pyrimidine or Pyrimido[4,5-d]pyrimidine moiety as described in U.S. Provisional Application No. 60/278032 filed Mar. 22, 2001 with the title “Pyrido[2,3-d]Pyrimidine and Pyrimido[4,5-d]Pyrimidine Nucleosides”, which is also incorporated by reference herein.

The sugar portion of contemplated adjuvant compounds can be modified, for example, according to the conformationally locked and/or constrained nucleoside analogs as described in U.S. patent applications Ser. Nos. 09/569183 and 09/309742 (both incorporated by reference herein). Consequently, contemplated compounds may include a sugar portion that comprises heteroatoms other than oxygen (e.g., sulfur, selenium, etc.), various substituents, and/or functional groups.

It should generally be appreciated that where contemplated compounds are effective to modulate the Type 1- Type 2 balance towards a Type 1 response, the response of the immune system towards an immunogen will generally increase a T-cell activated response. On the other hand, where contemplated compounds are effective to modulate the Type 1-Type 2 balance towards a Type 2 response, the response of the immune system towards an immunogen will generally increase a B-cell activated response. For example, Ribavirin, Levovirin, and Viramidine are known to stimulate a Type 1 response, while 7-(β-D-ribofuranosyl)pyrrolo[2,3-d]4-pyrimidone-5-carboxamidine*HCl is known to stimulate a Type 2 response (see e.g., U.S. Pat. Nos. 6,130,326, 6,063,772, and 5,767,097, all to Tam and all incorporated by reference herein).

In a further particularly contemplated aspect, suitable adjuvant compounds may also include modified or unmodified CpG dinucleotides and/or CpG-containing oligonucleotides, wherein the modification of such di- and oligonucleotides may include methylation, phosphorylation, replacement of one or more heteroatoms in the base (e.g., pyridopyrimidine base in the cytosine) or sugar moiety (e.g., sulfo-sugar in the cytosine), and/or addition or modification of substituents in the base (e.g., 5-methylcytosine) or sugar moiety (e.g., 2′-azacytosine). CpG dinucleotides and/or CpG-containing oligonucleotides have been reported to synergistically increase a Th1 cytokine response when administered with alum or cholera toxin (J Immunol. 1998, 161:4463-4466, and J Immunol. 1998, 160: 870-876). Consequently, it is contemplated that a combination of contemplated adjuvants with CpG dinucleotides and/or CpG-containing oligonucleotides will synergistically increase aTh1 cytokine response in a system challenged with an antigen. While not wishing to be bound to a particular theory, it is contemplated that the synergistic increase is at least in part due to an increased expression of costimulatory molecules.

Formulations

Formulations containing contemplated adjuvant compounds for adjuvants are most conveniently administered by oral, intranasal, intramuscular, or subcutaneous injections or as sustained release compositions although other methods of administration are also suitable. It should be especially appreciated that oral delivery of at least some of the contemplated compounds is already well known in the art for the purpose of antiviral treatment. Where appropriate, specific formulations to prevent hydrolysis during digestion would be necessitated for oral formulation. Alternatively, contemplated compounds may be included in a prodrug form to achieve a particular pharmacological property (e.g., solubility, specificity towards a target organ, half-life time, etc.). There are numerous formulations for vaccines known in the art, and suitable formulations may be prepared by a person of ordinary skill in the art without undue experimentation.

In alternative aspects, formulations may be liquid injectables or solids, which can be taken up in suitable liquids as suspensions or solutions for injection. Suitable excipients are, for example, water, saline, dextrose, glycerol, ethanol, and so forth. Nontoxic auxiliary substances, such as wetting agents, buffers, or emulsifiers may also be added. A particularly useful excipient comprises effective amounts of detergents, such as, 0.05% sodium dodecyl sulfate (SDS), to assure solubility.

A variety of techniques are known in the art to effect long-term stability and slow release. For example, the stability and half life of contemplated adjuvant compounds may be enhanced by coupling contemplated compounds to a hydrophilic polymer (e.g., polyethylene glycol (PEG) or a polyoxyethylated polyol). PEG-complexes are particularly useful where single sustained action dose of contemplated adjuvant compounds are administered. Alternatively, contemplated compounds may be succinylated to achieve long-term stability and slow release (Succinylation reaction may occur by contacting the contemplated adjuvant compounds with succinic anhydride, preferably at pH 5-9 and at room temperature in an aqueous solution containing a buffer with a solubilizing agent such as sodium dodecyl sulfate).

Sustained and continuous release formulations are of considerable variety, as is understood by those skilled in the art. An exemplary composition for sustained release parenteral administration is an injectable microcapsule formulation that with a single injection will deliver contemplated adjuvant compounds. Microcapsule formulation is typically a free-flowing powder consisting of spherical particles 20 to 100μm in diameter that can be resuspended in an appropriate vehicle and injected intramuscularly or subcutaneously with a conventional hypodermic needle (e.g., Microcapsules may comprise 0.5% to 5% of contemplated adjuvant compound encapsulated in poly(DL-lactide-co-glycolide) (DL-PLG) excipient, a biodegradable, biocompatible polyester).

With respect to the antigen, it is contemplated that all known antigens are considered suitable. However, particularly contemplated antigens include viral, bacterial, and synthetic (e.g., recombinant or by other preparation) antigens. For example, viral antigens include attenuated viruses and virus proteins (HIV, HBV, HCV, RSV, etc.), while bacterial antigens include bacterial cells, cell wall extracts, and bacterial proteins.

Administration

Contemplated adjuvant compounds will normally be administered separately from the vaccine, although it may, in some instances, especially in sustained or continuous release forms, be administered in combination with the vaccine. For example, where contemplated compounds are administered separately from the antigen, administration may occur prior to or after presentation of the antigen to the immune system. On the other hand, where contemplated adjuvant compounds are combined with the vaccine, the composition administered may contain an immunogen (i.e. antigen) that is effective in eliciting a specific response to a given pathogen or antigen, a pharmaceutically acceptable vaccine carrier and an immunopotentiating amount of contemplated compounds.

It is generally preferred to administer contemplated adjuvant compounds continuously (e.g., 5 to 30 days, preferably 5 to 14 days) dosages to achieve a plasma concentration of contemplated compounds effective for modulation of the Type 1 to Type 2 balance. Thus, administration of contemplated compounds may precede, overlap, and/or be after the immune system has been presented with the antigen against which immunity or enhanced immune reaction is desired. However, various alternative administrations are also contemplated, including discontinuous administrations. Suitable protocols for administration are disclosed, for example, in U.S. Pat. No. 6,150,337 to R. Tam, which is incorporated by reference herein.

Thus, specific embodiments and applications of nucleoside vaccine adjuvants have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, utilized, or combined with other elements, components, or steps that are not expressly referenced.

What is claimed is:

1. A pharmacological composition, comprising an antigen and an adjuvant, wherein the adjuvant comprises a nucleoside that changes expression of a Type 1 cytokine and a Type 2 cytokine of a human T-cell when the nucleoside analog is administered at a concentration effective to change the expression, and wherein the nucleoside is not an 8-substituted guanine nucleoside.

2. The composition of claim 1 wherein the antigen is selected from the group consisting of a viral antigen, a bacterial antigen, and a tumor-specific antigen.

3. The composition of claim 1 wherein the nucleoside comprises a nucleoside analog selected from the group consisting of Ribavirin, Levovirin, and Viramidine.

4. The composition of claim 1 wherein the modulation of the balance comprises a relative increase in the Type 1 response over the Type 2 response.

5. The composition of claim 1 wherein the composition further comprises at least one of a CpG-dinucleotide and a CpG-containing oligonucleotide.

6. The composition of claim 1 where the antigen is formulated for administration in a first route, and wherein the adjuvant is formulated for administration in a second route.

7. The composition of claim 6 wherein the first route includes injection and wherein the second route includes oral administration.

8. A method of immunizing a human, comprising:
providing a pharmacological composition comprising an antigen and an adjuvant;
wherein the adjuvant comprises a nucleoside that changes expression of a Type 1 cytokine and a Type 2 cytokine of a human T-cell when the nucleoside analog is administered at a concentration effective to change the expression, and wherein the nucleoside is not an 8-substituted guanine nucleoside; and
administering the pharmacological composition to the human.

9. The method of claim 8 wherein the antigen is selected from the group consisting of a viral antigen, a bacterial antigen, a tumor-specific antigen, an allergen, and a parasitic antigen.

10. The method of claim 8 wherein the nucleoside comprises a nucleoside analog selected from the group consisting of Ribavirin, Levovirin, and Viramidine.

11. The method of claim 8 wherein the modulation of the balance comprises a relative increase in the Type 1 response over the Type 2 response.

12. The method of claim 8 wherein the adjuvant additionally comprises at least one of a CpG-dinucleotide and a CpG-containing oligonucleotide.

13. The method of claim 8 wherein the step of administering includes administration of the antigen in a first route and administration of the adjuvant in a second route.

14. The method of claim 13 wherein the first route includes injection and wherein the second route includes oral administration.
